(19) United States
(12) Patent Application Publication
(10) Pub. No.: US 2023/0346888 A1
(43) Pub. Date: Nov. 2, 2023

(54) METHOD AND COMPOSITION OF TREATMENT OR PREVENTION OF CORONAVIRUS INFECTION

(71) Applicant: AINOS INC. TAIWAN BRANCH (USA), New Taipei City (TW)

(72) Inventors: Tsung-Fu YU, Taichung City (TW); Chun-Hsien TSAI, New Taipei City (TW); Ting-Chuan LEE, New Taipei City (TW); Chun-Jung TSAI, New Taipei City (TW)

(73) Assignee: AINOS INC. TAIWAN BRANCH (USA), New Taipei City (TW)

(21) Appl. No.: 18/140,535

(22) Filed: Apr. 27, 2023

Related U.S. Application Data
(60) Provisional application No. 63/336,052, filed on Apr. 28, 2022.
(60) Provisional application No. 63/354,975, filed on Jun. 23, 2022.
(60) Provisional application No. 63/375,962, filed on Sep. 16, 2022.

(51) Int. Cl. A61K 38/21; A61K 9/00; A61K 45/06; A61P 31/14

(52) U.S. Cl. CPC A61K 38/212; A61K 9/006; A61K 45/06; A61P 31/14

(57) ABSTRACT

The present application provides a method for treating and/or preventing an infection of Coronavirus comprising: providing a therapeutically effective amount of a composition comprising interferon to a subject via sublingual administration and/or buccal administration; wherein the Coronavirus comprises SARS-CoV-2.

### BACKGROUND OF THE INVENTION

### 1. Field of the Invention

The present invention relates to a method and a composition of treatment or prevention of Coronavirus infection.

### 2. Description of the Related Art

Coronavirus disease (COVID-19) is an infectious disease caused by the severe acute respiratory syndrome coronavirus 2 (SARS-CoV-2 virus). COVID-19 affects different people in different ways. Most infected people will develop mild to moderate illness and recover without hospitalization. However, in severe cases, the patients may rapidly progress acute respiratory distress syndrome (SARS), acute fatal lung failure, and/or multiple complicated multiple organ dysfunction syndrome. Almost all patients have a different degree of lung injury.

The COVID-19 infection inhibits the secretion of autologous interferon in lung cells, causing the continuous release of pro-inflammatory cytokines, resulting in the continuous accumulation of immune cells in the lungs, causing severe inflammation (i.e., cytokine storm) and even necrosis. Most of the current therapies use viral suppression or immunosuppression as the main mechanism of treatment.

Interferons (IFNs) are proteins made by host cells in response to the presence of pathogens such as viruses, bacteria, parasites or tumor cells. IFNs allow for communication between cells to trigger the protective defenses of the immune system that eradicate pathogens or tumors. Of the 9 different families of human interferon that have been identified, interferon-alpha (IFNα) is the most widely studied. The US FDA has approved the use of IFNα in mega doses given by injection for treating several cancers as well as hepatitis B and C. In the relevant technical field of interferon, the conventional knowledge is to use high dosages of interferon, for example, more than 1 million IU, and typically administered by intramuscular injection to systemically treat symptoms.

There is still a need for methods and/or compositions of treatment and/or prevention of COVID-19.

### SUMMARY

The present application describes a method for treating and/or preventing an infection of Coronavirus comprising: providing a therapeutically effective amount of a composition comprising interferon to a subject via sublingual administration and/or buccal administration; wherein the Coronavirus comprises SARS-CoV-2.

The present application also provides a composition for treatment and/or prevention of an infection of Coronavirus comprises a therapeutically effective amount of interferon alpha (INF-α), wherein the composition is in a dosage form of lozenge. The composition can be administered sublingually to a patient.

### BRIEF DESCRIPTION OF THE DRAWINGS

FIG. 1 to FIG. 6 show the body weight change in different animal experiments of the present application.

FIG. 7 and FIG. 8 respectively shows the viral titer determination of SARS-CoV-2 delta variant for the nasal turbinates and lung.

FIG. 9 and FIG. 10 respectively shows the viral titer determination of SARS-CoV-2 omicron variant for the nasal turbinates and lung.

FIG. 11A and FIG. 11B respectively shows the Histopathology Finding Table for the placebo group and the Veldona group in one animal experiment of SARS-CoV-2 delta variant.

FIGS. 12A-12D show the Histopathology Finding Table in one animal experiment of SARS-CoV-2 delta variant.

FIG. 13 and FIG. 14 show the photographs of pathology examination in one animal experiment of SARS-CoV-2 delta variant.

FIGS. 15A-15D show the Histopathology Finding Table in one animal experiment of SARS-CoV-2 omicron variant.

FIG. 16 and FIG. 17 show the photographs of pathology examination in one animal experiment of SARS-CoV-2 omicron variant.

### DETAILED DESCRIPTION OF THE EMBODIMENTS

In one aspect, the present application provides a method for treating and/or preventing an infection of Coronavirus comprising: providing a therapeutically effective amount of a composition comprising interferon to a subject via sublingual administration and/or buccal administration; wherein the Coronavirus comprises SARS-CoV-2.

The Coronavirus can be severe acute respiratory syndrome coronavirus 2 (SARS-CoV-2), SARS-CoV-1, Middle East respiratory syndrome-related coronavirus (MERS-CoV) and the like. In one embodiment, the Coronavirus is SARS-CoV-2. Clinically, the SARS-CoV-2 has several variants such as Alpha (B.1.1.7), Beta (B.1.351), Gamma (P.1), Delta (B.1.617.2), Mu (B.1.621), R.1, Epsilon, Theta, and Zeta. The present treatment and/or prevention method can be applied to all variants of the SARS-CoV-2.

The method of the present application is characterized by the sublingual administration and/or buccal administration of the composition comprising the interferon. Examples of suitable interferon can include type I INFs, type II INFs, and type III INFs.

In preferable embodiments, the composition comprises interferon alpha (INF-α). The INF-α can be a native INF-α, a recombinant INF-α, or a mixture thereof. In a preferred embodiment, the INF-α contained is recombinant INF-α.

In some embodiments, the composition can further comprise interferon beta and/or interferon gamma.

In contrast to the conventional knowledge, the present application applies a composition having low-dose IFNα for oral delivery, a non-toxic alternative to high-dose injectable IFNα. More particularly, the low-dose IFNα is via sublingual administration and/or buccal administration.

Sublingual administration involves placing the composition under the tongue to dissolve and absorb into the subject's blood through the tissue under the tongue. Buccal administration involves placing the composition between the gums and cheek, where the composition dissolves and is absorbed into subject's blood. Generally, the composition administered sublingually and/or buccally does not pass through and adsorbed by the digestive tract.

Examples of the dosage forms of sublingual administration and/or buccal administration can include, but not be limited to, lozenge, tablet, film, spray and the like. In one embodiment, the composition is in the dosage form of lozenge. In one embodiment, the composition is in the dosage form of orally disintegrating tablet. The orally disintegrating tablet is able to disintegrate in the mouth within seconds without the need for additional liquid.

Accordingly, via sublingual administration and/or buccal administration, the composition can be quickly contacted and absorbed by oral mucosa, and the therapeutic effects can be achieved more efficiently. In addition, higher drug compliance can be developed by sublingual administration and/or buccal administration.

In one embodiment, the composition comprises a dosage of equal to or less than 1,000 International Units (IU) of INF-α. In one embodiment, the composition comprises a dosage of equal to or less than 1,000 microgram of INF-α. The dosage can be, for example, less than 900 IU, less than 800 IU, less than 700 IU, less than 600 IU, less than 500 IU, less than 400 IU, less than 300 IU, less than 200 IU, or less than 100 IU.

In one embodiment, the composition comprises a dosage of equal to or more than 1 IU of INF-α, for example, more than 2 IU, more than 3 IU, more than 4 IU, more than 5 IU, more than 10 IU, more than 25 IU, more than 50 IU, more than 75 IU, more than 80 IU, or more than 90 IU.

In one embodiment, the composition comprises a dosage of 1 IU -1,000 IU of INF-α, for example, 2 IU, 3 IU, 4 IU, 5 IU, 8 IU, 10 IU, 25 IU, 50 IU, 75 IU, 80 IU, 90 IU, 100 IU, 200 IU, 300 IU, 400 IU, 500 IU, 600 IU, 700 IU, 800 IU, 900 IU, 950 IU, or a value falling within the scope between any two of the above values.

In a preferred embodiment, the INF-α contained in the composition can be 1 IU-500 IU. In another preferred embodiment, the INF-α contained in the composition can be 1 IU-100 IU. In another preferred embodiment, the INF-α contained in the composition can be 5 IU-50 IU. In a more preferred embodiment, the INF-α contained in the composition can be 1 IU-10 IU.

The composition can comprise a buffer, a carrier, an excipient or the like.

Examples of suitable buffers can include, but not be limited to, acetate, phosphate, citrate, borate and the like. In some embodiments, the buffer can be phosphate buffered saline (PBS), saline, and the like.

Examples of suitable carriers can include, but not be limited to, carbohydrates, antioxidants, chelating agents, low molecular weight proteins and the like. In some embodiments, the carriers can be glucose, sucrose, dextrans, ascorbic acid, glutathione and the like.

Examples of suitable excipients can include, but not be limited to, diluents, coating agents, coloring agent, lubricants, preservatives, flavors and the like. In some embodiments, the excipients can be ethanol, lactose, starch and the like.

In one embodiment, the method for treating and/or preventing an infection of Coronavirus further comprises: administering the subject with a therapeutically effective amount of an antiviral agent, anti-inflammatory agent, interferon beta and/or interferon gamma. Examples of the antiviral agents can include, but not be limited to, remdesivir, Paxlovid, Molnupiravir, Xocova and the like. Such administration can be prior to, simultaneously with, or after the administration of the composition comprising INF-α. The route of such administration are not limited, for example, the antiviral agents can be administered orally or via injection or inhalation.

In another aspect, the present application also provides a composition for treatment and/or prevention of an infection of Coronavirus comprises a therapeutically effective amount of interferon alpha (INF-α), wherein the composition is in a dosage form of sublingual administration and/or buccal administration.

In one embodiment, the INF-α is a recombinant INF-α.

In one embodiment, the INF-α is 1 IU-1,000 IU, for example, 2 IU, 3 IU, 4 IU, 5 IU, 8 IU, 10 IU, 25 IU, 50 IU, 75 IU, 80 IU, 90 IU, 100 IU, 200 IU, 300 IU, 400 IU, 500 IU, 600 IU, 700 IU, 800 IU, 900 IU, 950 IU, or a value falling within the scope between any two of the above values.

In one embodiment, the composition further comprises a buffer, a carrier and/or an excipient. In some embodiments, the buffer can be phosphate buffered saline (PBS).

In one embodiment, the composition further comprises interferon beta and/or interferon gamma.

In one embodiment, the composition further comprises an antiviral agent and/or anti-inflammatory agent.

In the present application, the dosage form of sublingual administration and/or buccal administration can include lozenge, tablet, film, spray and the like. In one embodiment, the composition is in the dosage form of lozenge.

In the present application, the composition is delivered into the oral cavity as a lozenge in low doses, i.e. 1-1000 IU, of INF-α. Orally administered IFNα is able to activate dozens of immune system genes in the peripheral blood, and can be effective against viral diseases without the side effects associated with high-dose injections of IFNα.

In the present application, the sublingual administration and/or buccal administration of the low-dose IFNα can be quickly adsorbed by the subject, prevent the decrease of therapeutic effects caused by digestion, and develop higher drug compliance.

### EXAMPLES

The Golden Syrian hamster model is recommended by BARDA (Biomedical Advanced Research and Development Authority, Health and Human Services, US) as an in vivo efficacy indicator as it can be infected without artificial genetic engineering. In practice, hamster will recover from infection in 7-10 days and the general clinical sign is weight loss (10-20%) with no fever. In the present application, the drug efficacy on viral eradication is to be evaluated via the primary indicator of viral loads, in nasal and in lung. Also, the percentage of body weight change during the infection. For exploring the possible mechanism of drug action, the secondary indicator to assess the plasma levels of several key cytokines with qPCR is also proposed.

In the following Examples, male golden hamsters were obtained from the National Laboratory Animal Center (National Applied Research Laboratories, Taiwan). All experiments were performed at the ABSL-3 core facility, (Institute of Preventive Medicine, National Defense Medical College). The hamsters were randomized from different litters into experimental groups and were acclimatized at the ABSL-3 facility for one week before the experiments. The study protocol was reviewed and approved by the Committee on the Institutional Animal Care and Use, Institute of Preventive Medicine (Permit number:AN-111-11).

### Example 1

### Materials and Methods

Cell Line and Virus

Vero E6 cells were cultured in DMEM medium with 10% fetal bovine serum. Cell passage numbers in all cases were less than 25. SARS-CoV-2 virus was provided by Taiwan Center for Disease Control (hCoV-19/Taiwan/1144/2021; B.1.617.2; EPI_ISL_5854263) and amplified in Vero E6 cells. SARS-CoV-2 titers were determined via plaque assays and processed in BSL-3 or BSL-4 laboratories.

SARS-CoV-2 delta variant and omicron variant were applied in the present application.

Animal Experiments

12 hamsters (˜5-6 weeks old) were challenged with SARS-CoV-2 (delta variant) (dose: 1×10⁴ pfu/ml) via intranasal administration (50 ul/nare) at Day 0. Then the hamsters were divided into two groups, in which one group were administered with the composition of the present application (sometimes briefly shown as “Veldona group” hereafter), and the other group were administered with a buffer solution as the placebo group (also called “control group” hereafter). In this example, the composition of the present application having interferon alpha with a dose of 8 IU/100 g (QD). At this dose for hamsters, no toxicity is found. The hamsters were sacrificed for the following analysis.

For the test of SARS-CoV-2 delta variant, the hamsters were administered the composition of the present application daily after 6 hours of virus infection on Day 0. The hamsters were sacrificed on Day 3 and Day 6.

For the test of SARS-CoV-2 omicron variant, the hamsters were administered daily before virus challenge for 5 days (Day −5 to Day −1) and after 6 hours of virus infection on Day 0. The total period of treatment was 16 days. The hamsters were sacrificed on Day 2, Day 5 and Day 10.

Body Weight Change

Body weight of the hamster was measured daily during the experiment period, and change of the body weight was calculated.

Lungs and Nasal Turbinates Processing Steps

Lung tissue was transferred to a 2 mL tube containing respectively 1 mL of DMEM medium and 3 mm glass beads. They were crushed using a Tissue Lyser machine (Bertin/Precellys24). 100 μL supernatant media (Lung RT-qPCR supernatant) was transferred to a 2 mL tube containing 0.9 mL of TRIzol reagent for RNA extraction. 100 μL supernatant media were ready to perform plaque assay. Both 250 μL supernatant media were stored at −80 ° C. for backup. The extraction of nasal turbinates RT-qPCR supernatant were follow the procedure of lung tissue processing.

Quantitative Real-Time RT-PCR (RT-qPCR) Assays

100 μL of tissues supernatant was taken for RNA extraction via TANBead Nucleic Acid Extraction kit. After RNA being quantified, 1 μg of RNA was reverse transcribed to cDNA using SuperScript IV Reverse Transcriptase kit (Invitrogen). Subsequently, RT-qPCR was performed with a PowerTrack SYBR Green Master Mix ((Applied biosystems) and the LightCycler 480 system using primer pairs specific for the NP gene. The RT-qPCR target genes included SARS-CoV-2 NP gene, IFN-α(−2b), IFN-β, IL-λ, TNF-α and TGF-β, and GAPDH as the internal control.

Pathological Section and Interpretation of Lung Tissue

Entrust the National Laboratory Animal Center to perform HE staining and interpretation of lung tissue pathological sections. The histopathological evaluation was performed on the submitted lungs. Severity of lesions (except inflammation area of the lung) was graded according to the methods described by Shackelford et al. (Toxicologic Pathology, Vol 30, No 1, pp 93-96, 2002). Degrees of lesions were graded histopathologically from zero to five depending on severity (0=not present; 1=minimal (<1%); 2=slight (1-25%); 3=moderate (26-50%); 4=moderately severe (51-75%); 5=severe/high (76-100%)).

Lesions (from left lobes) will be evaluated visually at necropsy and lung pictures will be taken as evidence of any gross pathology. Tissues (from left lobe) will be harvested in cassette for histopathology H&E stain.

Results

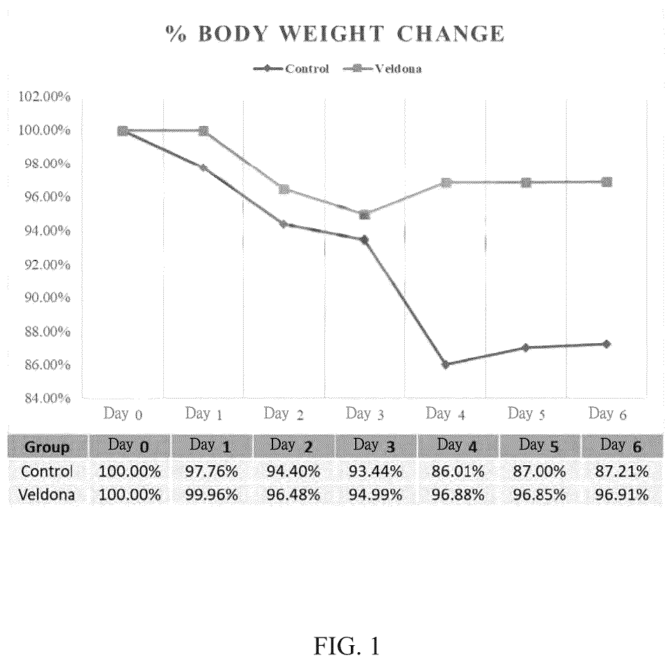

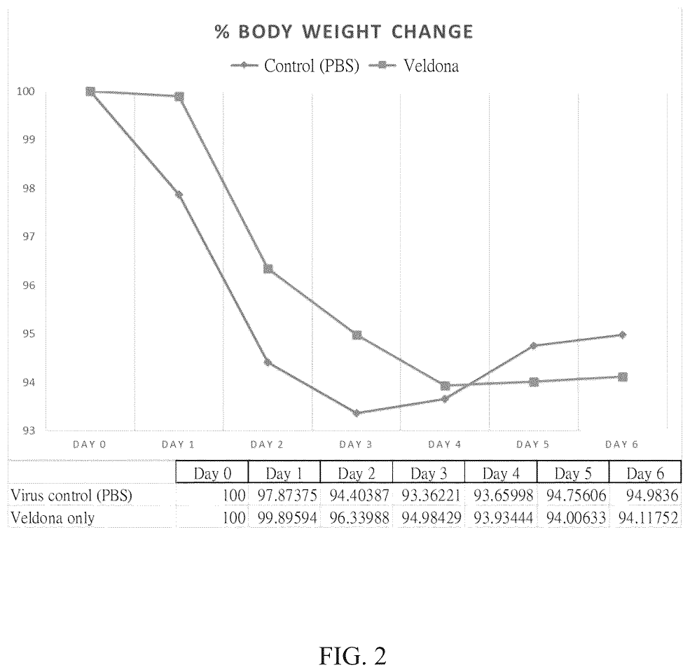

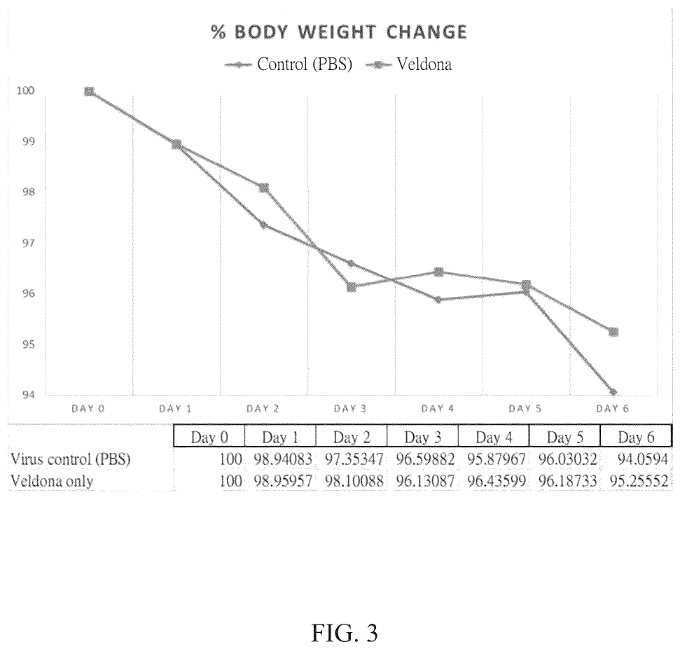

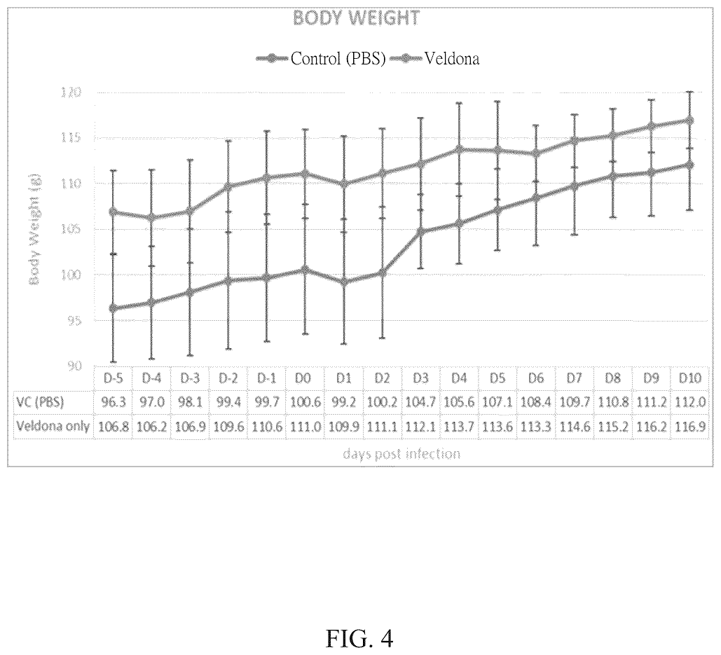

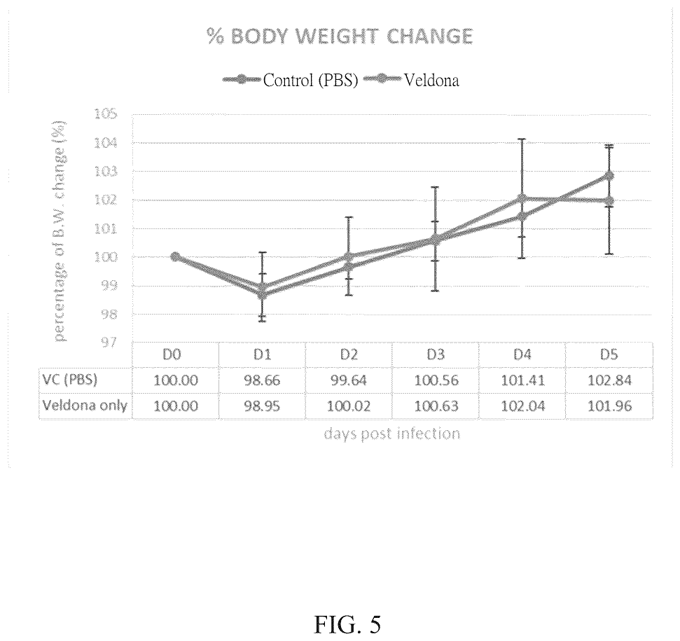

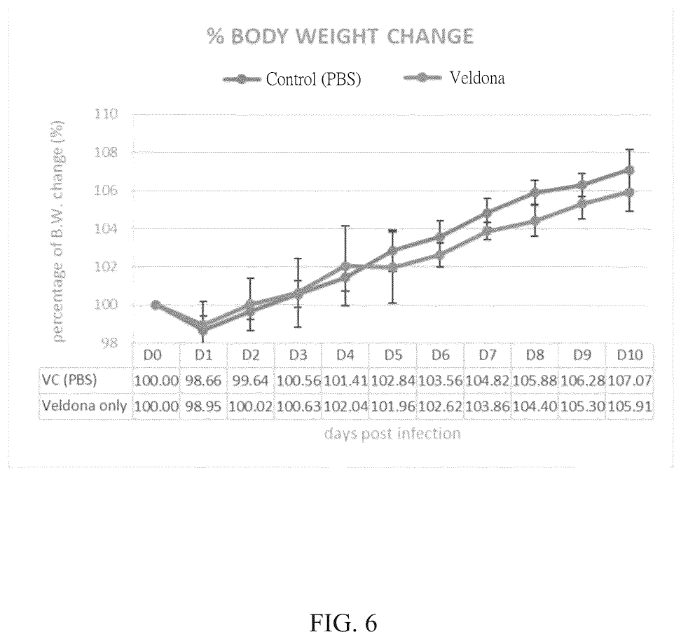

Several independent animal experiments were conducted, and the results were shown as follows. FIG. 1 to FIG. 6 show the percentage of body weight change in different animal experiments. FIGS. 1-3 were for SARS-CoV-2 delta variant while FIGS. 4-6 were for SARS-CoV-2 omicron variant. As shown in the figures, the hamsters were most severe in the first three days after being infected with the virus, and their body weight dropped rapidly. About 7-10 days after infection, the hamsters recovered and regained their weight. The results showed that compared with the control group, the hamsters in the Veldona group regained their body weight significantly at Day 4, and almost returned to the state before infection. Then the body weight remained stable. This shows that the composition of the present application has a significant anti-COVID-19 virus effect.

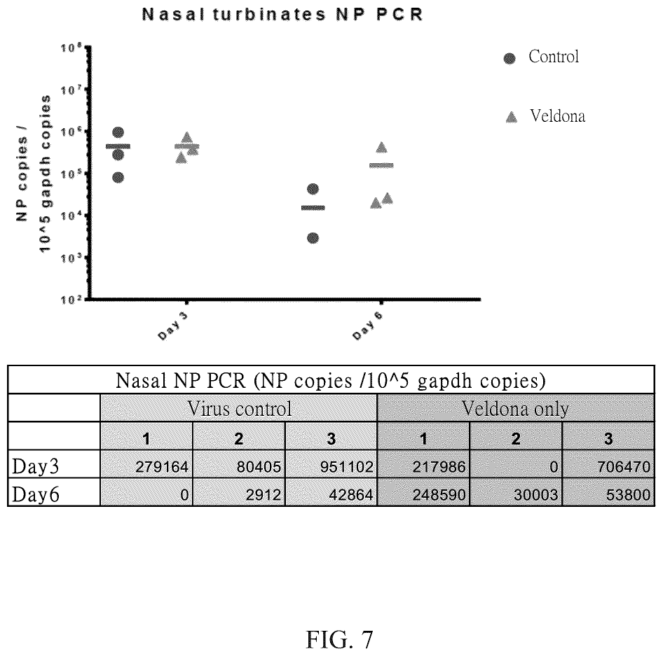

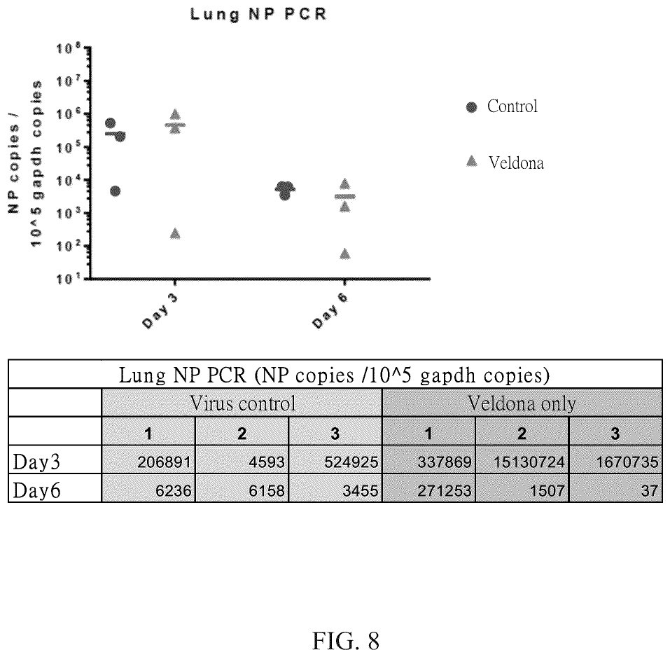

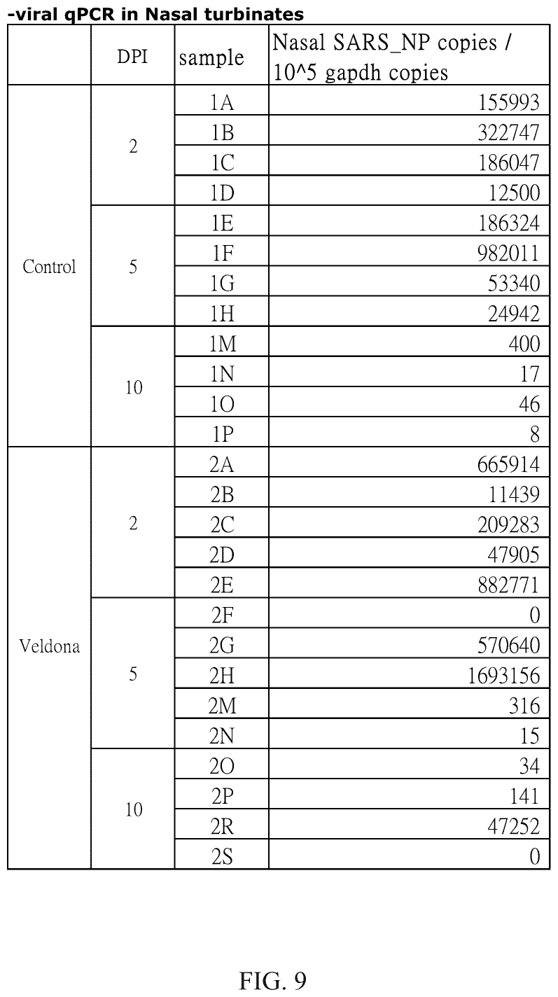

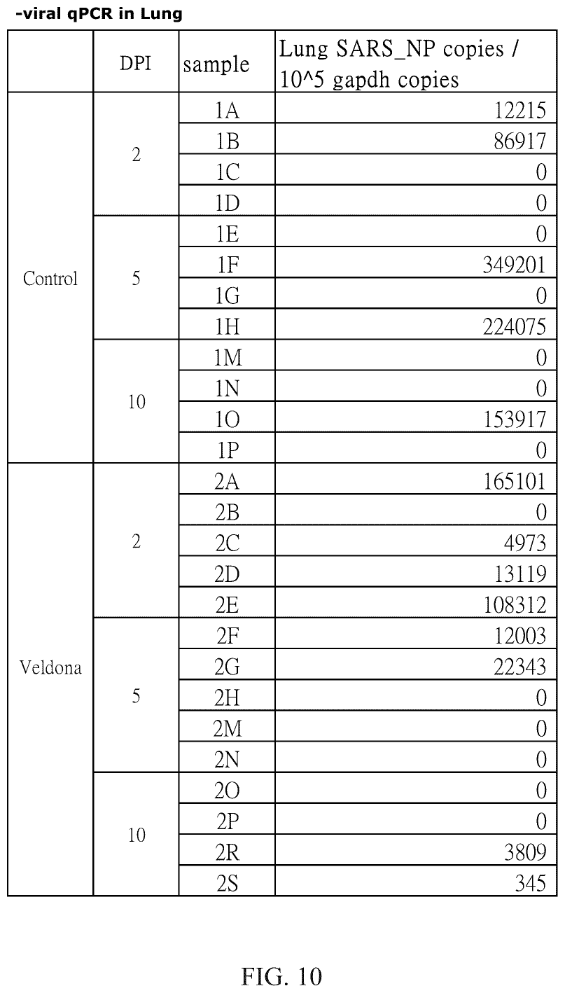

FIG. 7 and FIG. 8 shows the viral titer determination of SARS-CoV-2 delta variant for the nasal turbinates and lung, respectively. FIG. 9 and FIG. 10 shows the viral titer determination of SARS-CoV-2 omicron variant for the nasal turbinates and lung, respectively.

According to the results shown in FIG. 7 and FIG. 8, the amount of SARS-CoV-2 delta variant in the nasal cavity of the Veldona group on the third day after infection was similar to that of the control group, but on the sixth day after infection, it was found that the amount of virus in four hamsters was significantly reduced. More importantly, in the lower respiratory tract infection part, the Veldona group had less virus in the lungs of the hamsters on the third day after infection than the placebo group. On the sixth day after infection, it was found that hamsters had a significant reduction in the amount of virus.

According to the results shown in FIG. 9 and FIG. 10, the Veldona group significantly had less virus in the lungs of the hamsters on the fifth day after infection and no inflammation on the tenth day than the placebo group. On the tenth day after infection, it was found that the hamsters in the Veldona group had a significant reduction of viral loads in lungs. Combine the two results above, the hamsters in the Veldona group show good anti-COVID-19 efficacy during the 15 days treatment period.

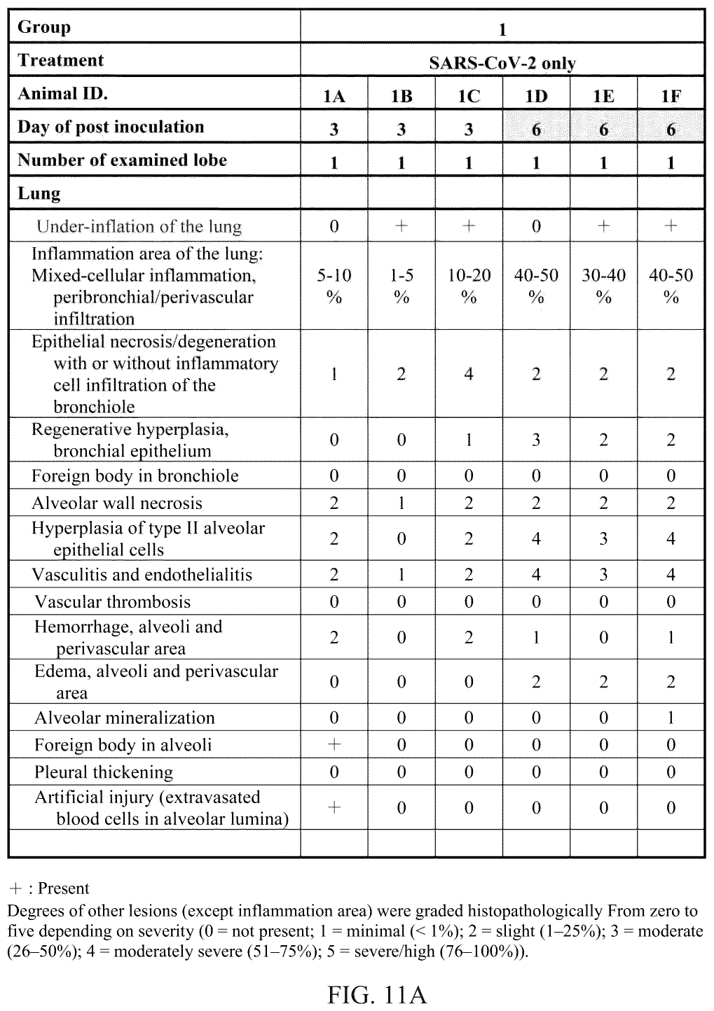

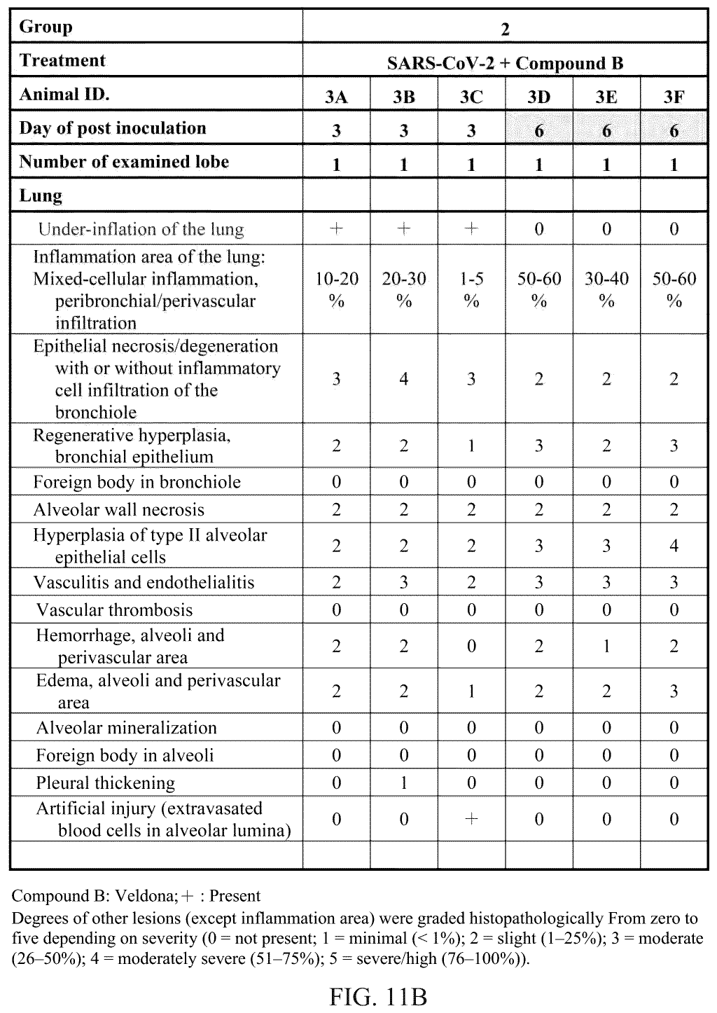

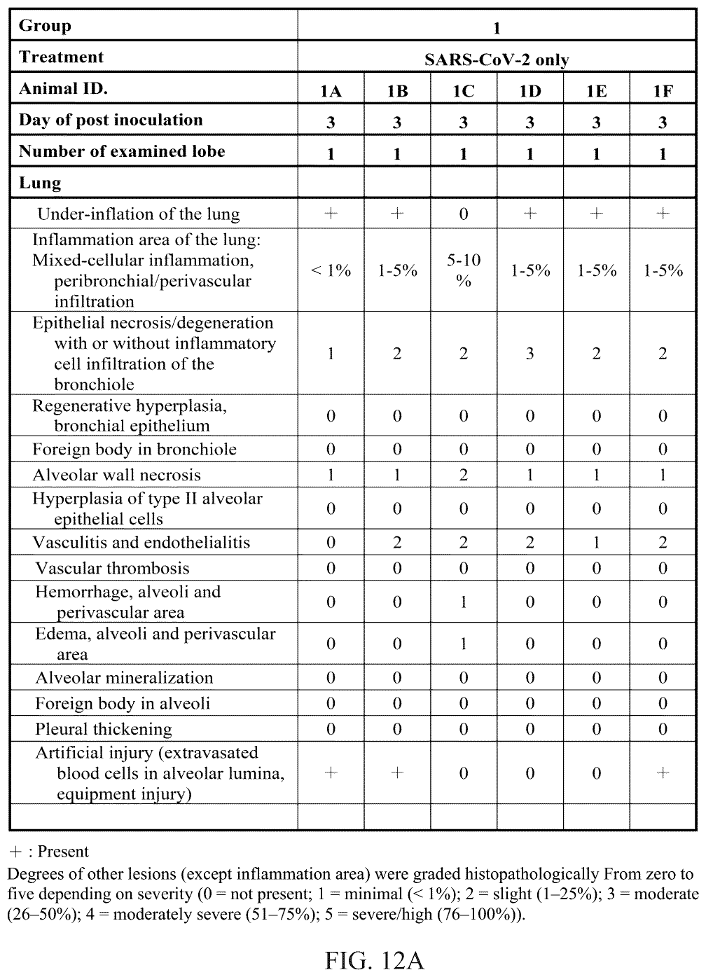

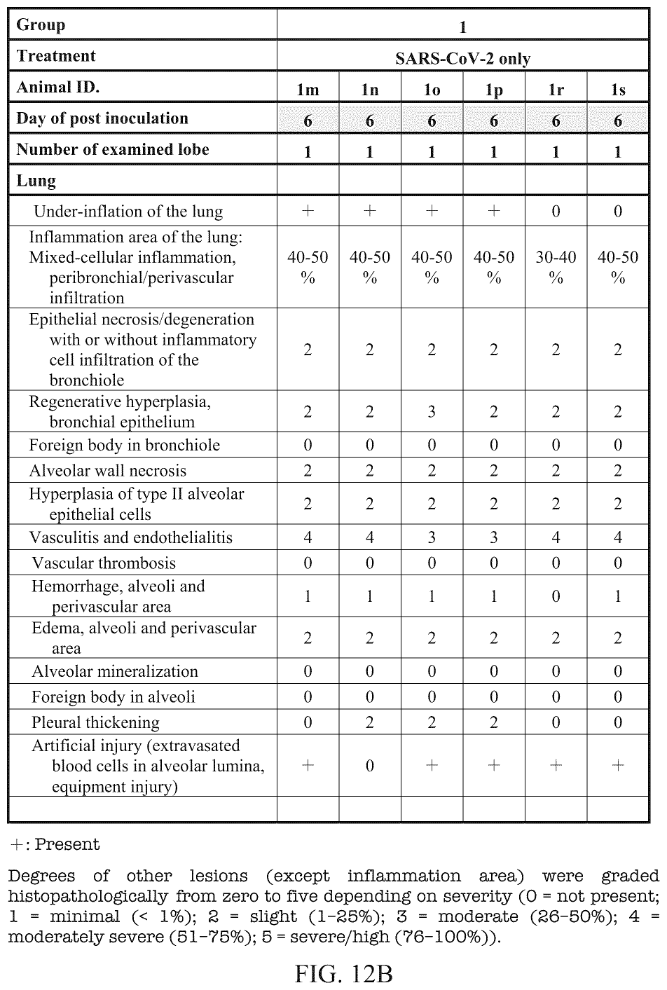

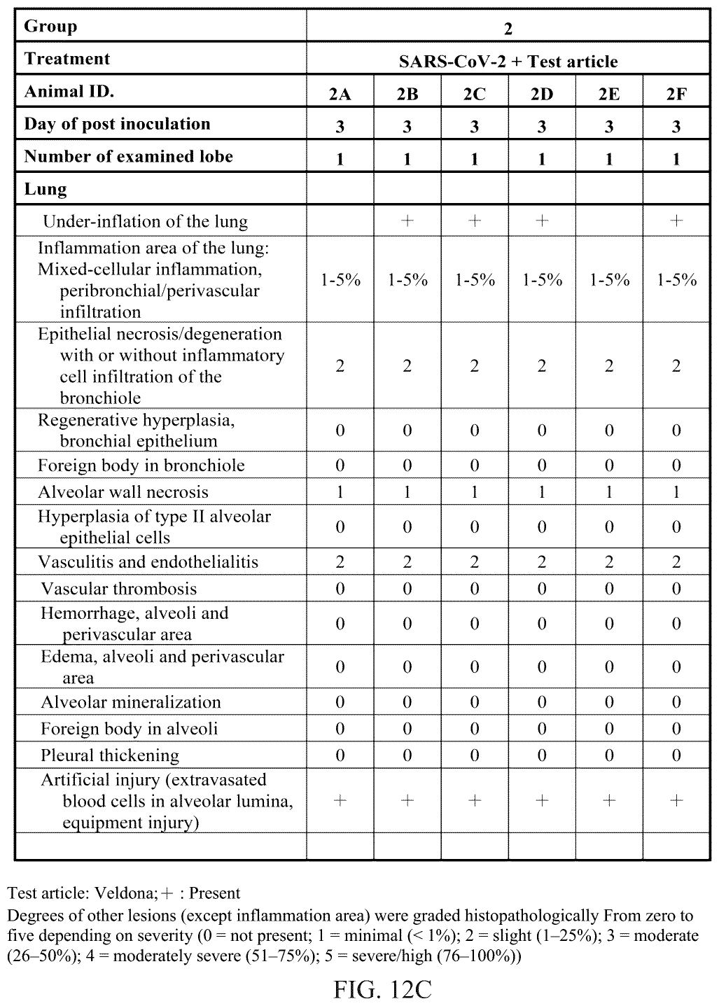

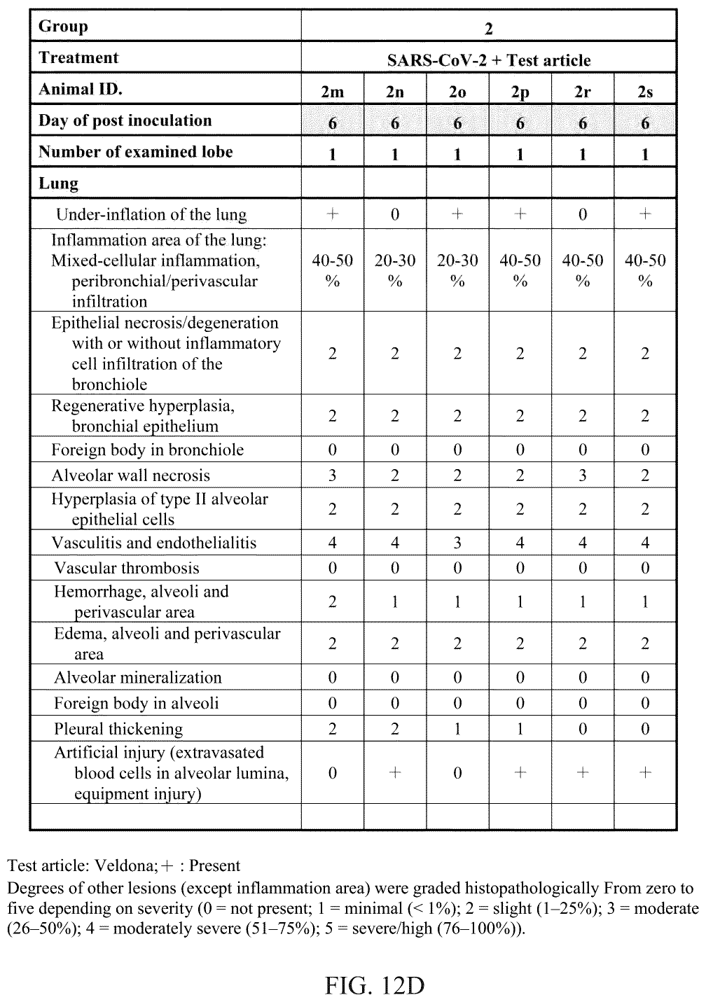

FIG. 11A and FIG. 11B respectively shows the Histopathology Finding Table for the placebo group and the Veldona group in one animal experiment of SARS-CoV-2 delta variant. FIGS. 12A-12D shows the Histopathology Finding Table in another animal experiment of SARS-CoV-2 delta variant.

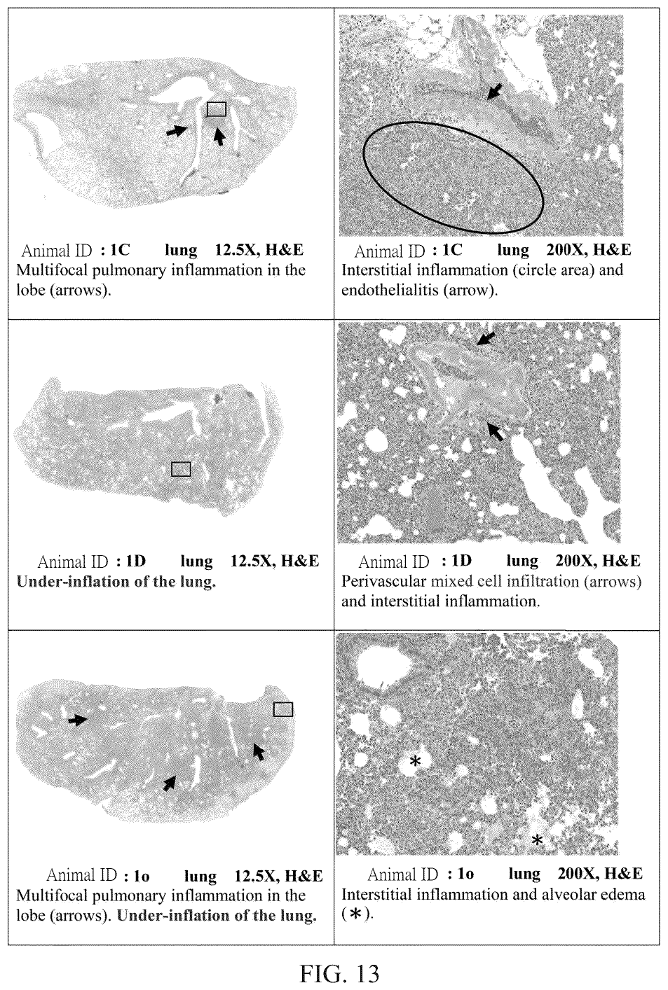

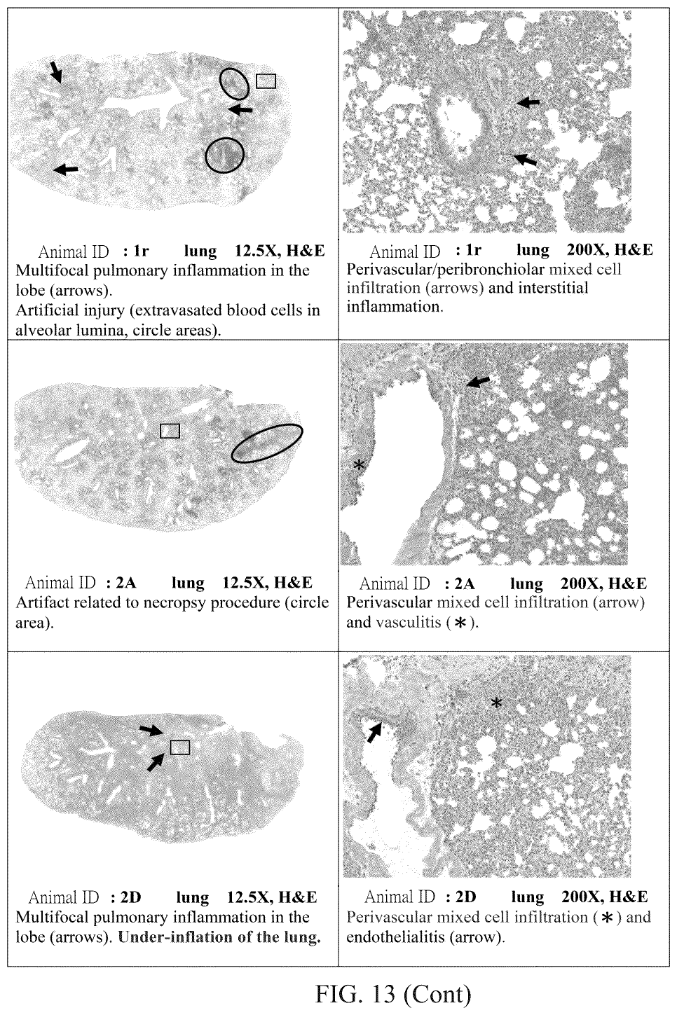

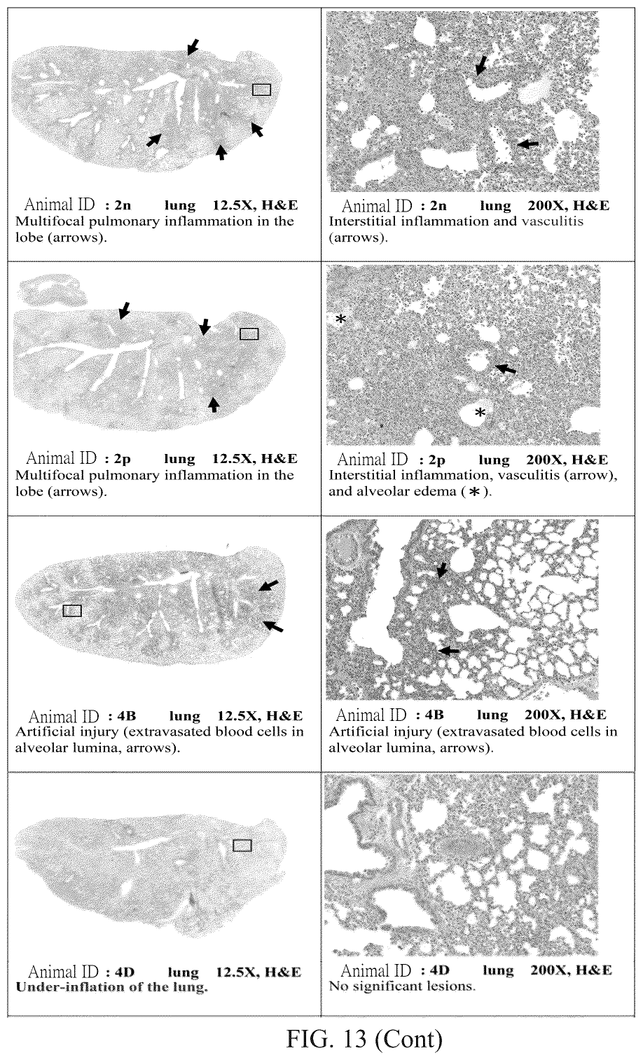

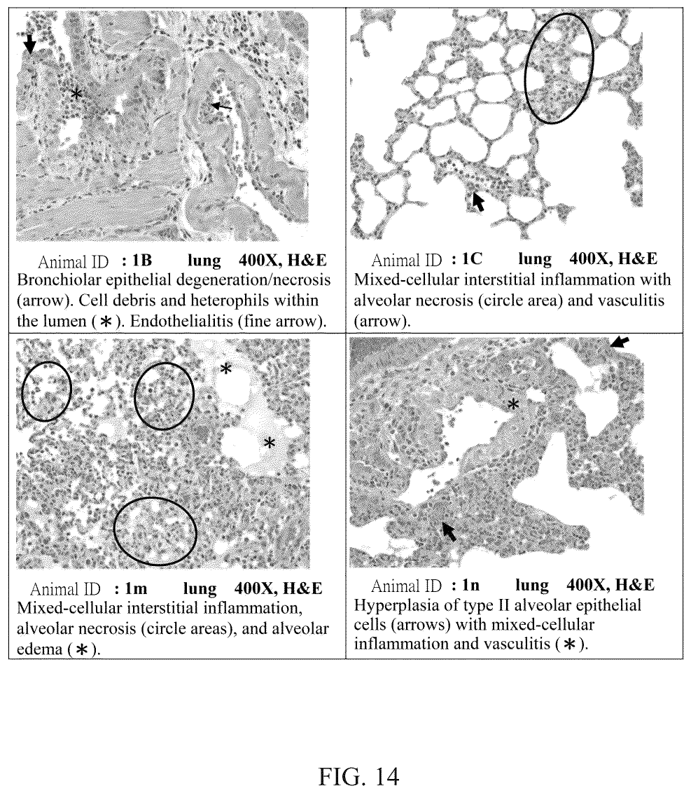

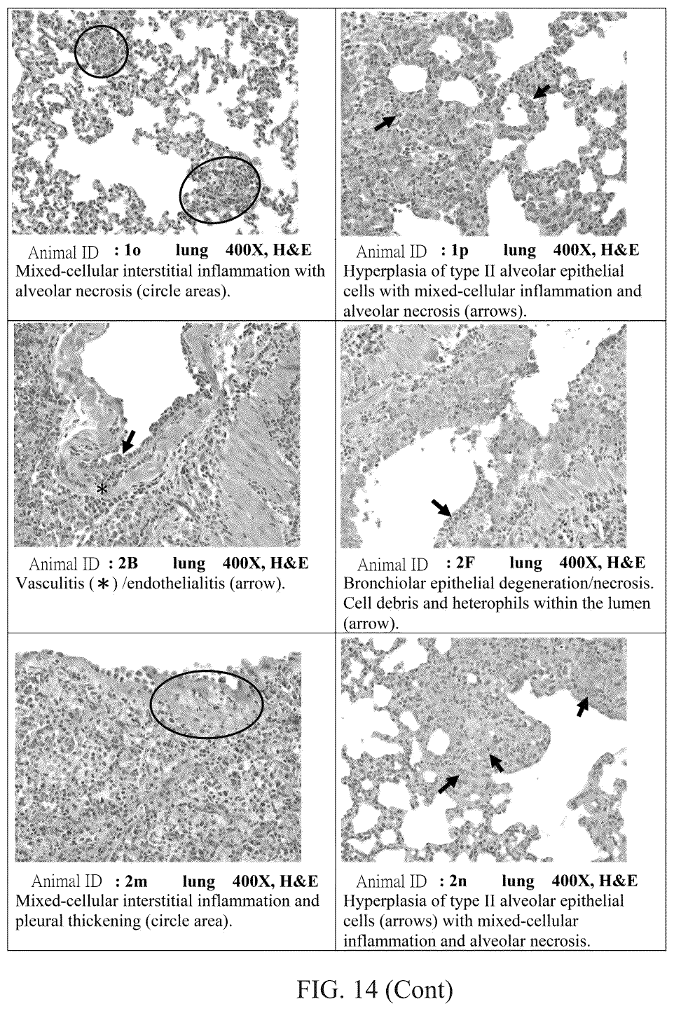

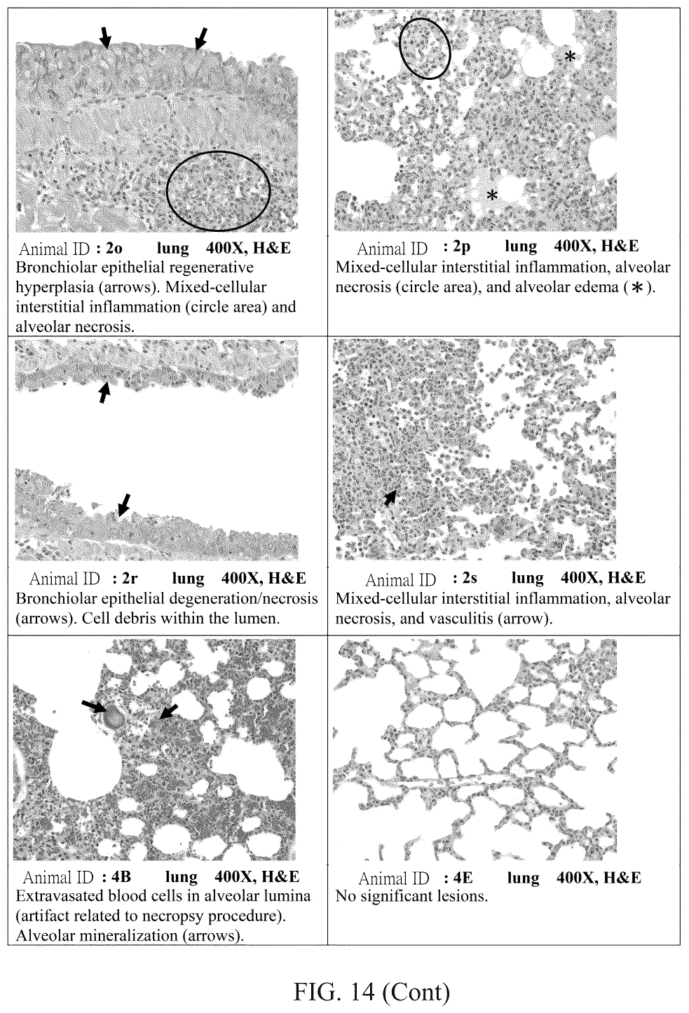

FIG. 13 and FIG. 14 show the photographs of pathology examination in one animal experiment of SARS-CoV-2 delta variant. The left column of FIG. 13 shows the lung tissue sections with H&E staining, the multifocal pulmonary inflammation in the lobe were indicated by arrows, and the artificial injury (extravasated blood cells in alveolar lumina) or artifact related to necropsy procedure were indicated by the circle areas. The right column of FIG. 13 shows the partial enlargement of the lung section, i.e. the higher magnification (200×) of the boxed area indicated in the left column. The animal ID and the observed symptoms were noted under each photograph. The animal ID and its detail information were shown in Table 1.

TABLE 1

| | | | | Number |
|---|---|---|---|---|
| | | | Day of post | of |
| Groups | Treatment | Animal ID. | inoculation | animals |
| 1 | SARS-CoV-2 delta | 1A, 1B, 1C, | 3 | 3 |
| | variant (placebo) | 1D, 1E, 1F | | |
| | | 1m, 1n, 1o, | 6 | 3 |
| | | 1p, 1r, 1S | | |
| 2 | SARS-CoV-2 delta | 2A, 2B, 2C, | 3 | 3 |
| | variant and Test | 2D, 2E, 2F | | |
| | article | 2m, 2n, 2o, | 6 | 3 |
| | | 2p, 2r, 2S | | |
| 3 | SARS-CoV-2 delta | 4A, 4B, 4C | 3 | 3 |
| | variant and | 4D, 4E, 4F | 6 | 3 |
| | Compound B | | | |

Test article: Veldona (8 IU); Compound B; Veldona (8 IU)

In one of the animal experiments of SARS-CoV-2 delta variant, the histopathological examination results included the microscopic alterations presented in Histopathology Finding Table of FIGS. 12A-12D and the pathological photos presented in FIGS. 13-14. Histologically, Syrian hamsters infected with SARS-CoV-2 (Delta variant) presented lesions including:

Mixed-Cellular Inflammation, Peribronchial Infiltration, and Perivascular Infiltration:

At 3 dpi (day of post inoculation), the lesions were characterized by a mixture of heterophils with lymphocytic and histiocytic cell types within alveoli/interstitial and peribrochial and perivascular in the lung. The lesion severity was minimal to slight. At 6 dpi, hamsters developed slight to moderate mixed-cellular interstitial inflammation of the lung, chiefly mononuclear cell with few heterophils.

Bronchial Epithelial Cell Degeneration/Necrosis with or without Inflammatory Infiltration in Bronchiole:

The lesions were observed in lungs of SARS-CoV-2 inoculated animals and characterized by cellular swelling, cytoplasmic vacuolation, perinuclear clear spaces, pyknosis of nuclei of affected epithelium mixing up with heterophils infiltrate into the bronchial lumen. The lesion severity was minimal to moderate.

Regenerative Hyperplasia of Bronchiolar Epithelium:

The lesion was predominantly observed in lungs of SARS-CoV-2 inoculated animals at 6 dpi. Regenerative hyperplasia of the epithelium is a common response to epithelial injury. Hyperplasia of the bronchiolar epithelium is characterized by increased layers of surface respiratory epithelial cells, usually lacking cilia. The lesion severity was slight.

Alveolar Wall Necrosis:

The lesion was observed in lungs of SARS-CoV-2 inoculated animals and characterized by cellular swelling and pyknosis of nuclei of affected epithelium. The lesion severity was minimal to slight.

Hyperplasia of Type II Alveolar Epithelial Cells:

The lesion was predominantly observed in lungs of SARS-CoV-2 inoculated animals at 6 dpi. Features of hyperplasia of type II alveolar epithelial cells showed karyomegaly, cytomegaly, and reactive hypertrophy with scattered syncytia. The lesion severity was slight.

Vasculitis and Endothelialitis:

The lesions were observed in lungs of SARS-CoV-2 inoculated animals.

Endothelialitis is characterized by blood vessel bulging into the lumen due to an infiltration by macrophages and lymphocytes. Inflammation of the vessels (vasculitis) is characterized by fibrinoid degeneration and inflammatory infiltrates of heterophils and lymphocytes. The lesion severity was minimal to moderately severe.

Hemorrhage and Edema of the Pulmonary Parenchyma:

Pulmonary hemorrhage is characterized by accumulations of extravasated blood cells in alveolar lumina. Pulmonary edema is characterized by accumulations of homogenous eosinophilic material in alveolar lumina. The lesions were observed in some submitted lungs. Hemorrhage and edema results from either alteration in pulmonary hemodynamics or damage to the air-blood barrier in alveolar walls. The lesion severity was minimal to slight. In addition, pleural thickening was observed in some submitted lungs. The lesion may be attributable to SARS-CoV-2 inoculated. Alveolar mineralization was observed in some submitted lungs. The lesion may be attributable to spontaneous or incidental lesion. Artificial injury related extravasated blood cells in alveolar lumina was considered as related to tissue sampling injury.

The pathological indicators that the Veldona group was better than the control group were all lung symptoms of COVID-19. For example, in the Veldona group, it could be observed that: (1) none of under-inflation of lung at 6 dpi, which symptom is a condition associated with pneumonia and could lead to acute respiratory distress syndrome (ARDS), shortness of breath, fatigue, difficulty inhaling, and exercise intolerance; (2) lower degree of vasculitis and endothelialitis, which could lead to thrombus; (3) less hyperplasia of type II alveolar epithelial cells. Under-inflation of lung, which could lead to pulmonary fibrosis and ARDS. ARDS patients usually need the intensive medical care and could cause 40-70% of mortality rate.

In this study, though the severity of pneumonia in left lobe of lung was no prominent differences in the Veldona group and the control group at 3 dpi and 6 dpi the Veldona group had lower expression of IL-6 and TNF-alpha and higher expression of IFN-alpha, IFN-gamma and IFN-lamda, which were confirmed by the cytokines qPCR results of the lung tissues. Obviously, the Veldona group had better anti-viral immune response without cytokine storm. According to the above results, the Veldona group with the moderate pneumonia (40-50%) had less viral load compared to the control group.

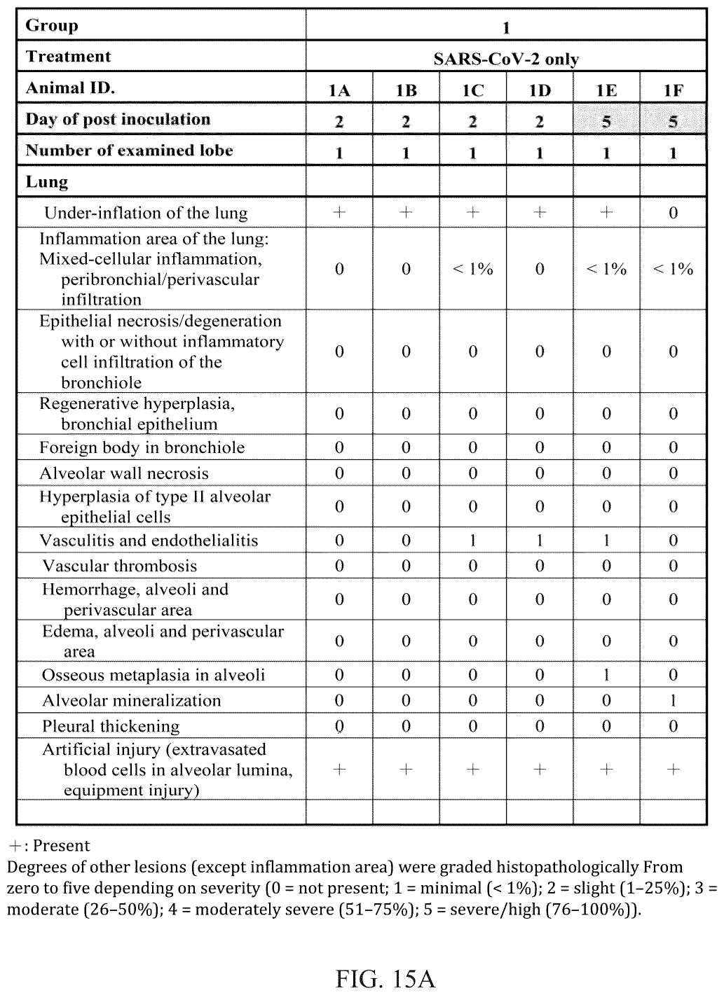

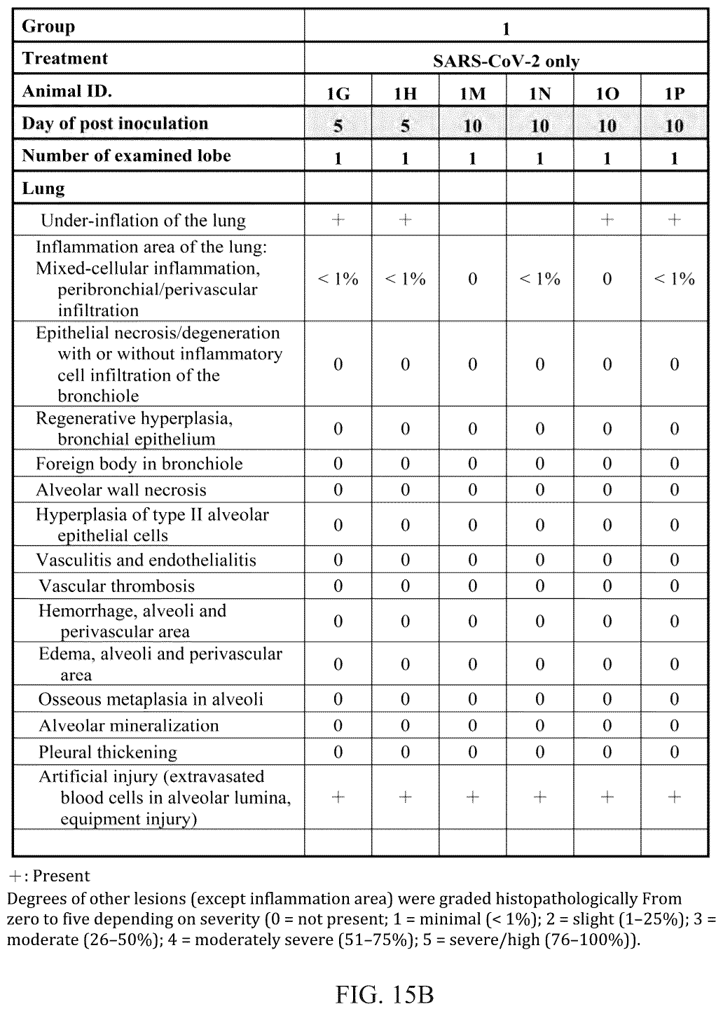

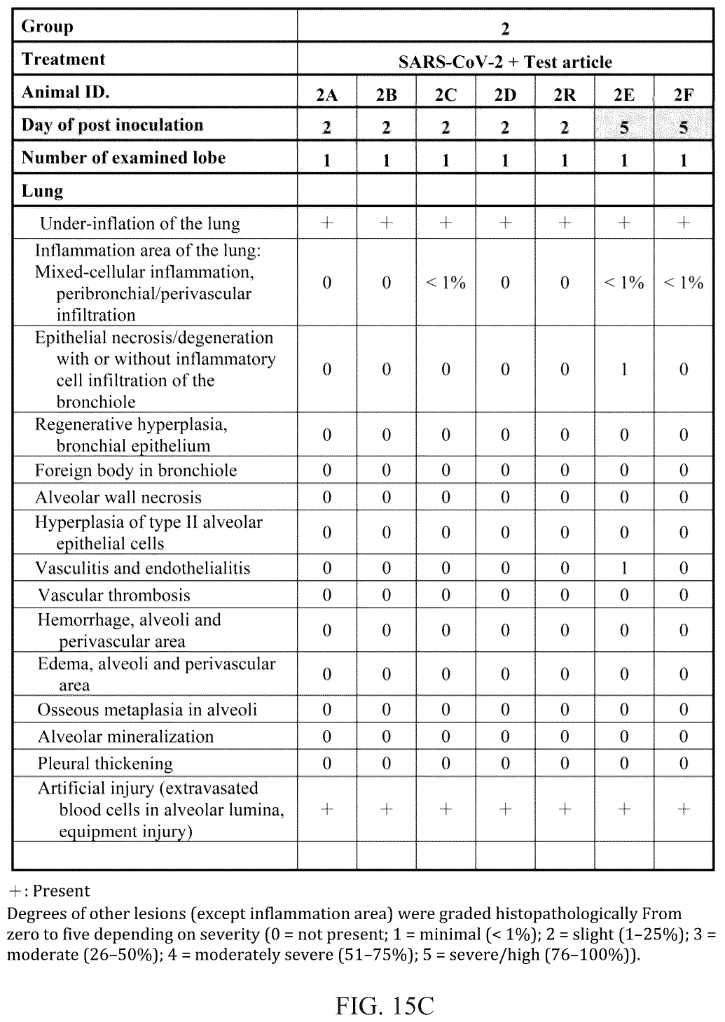

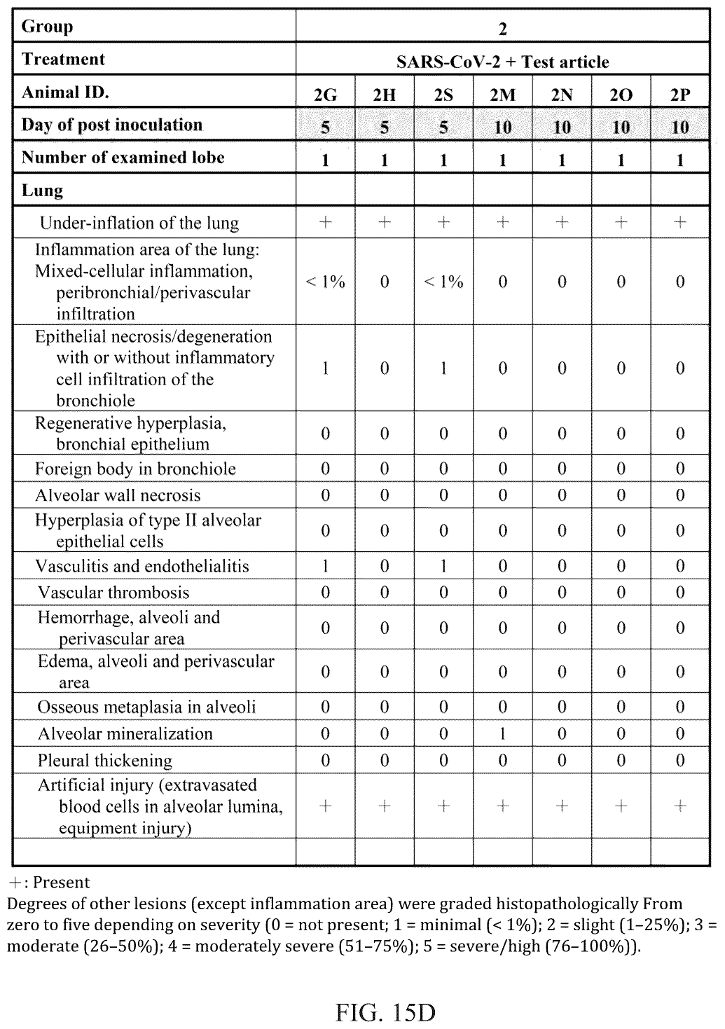

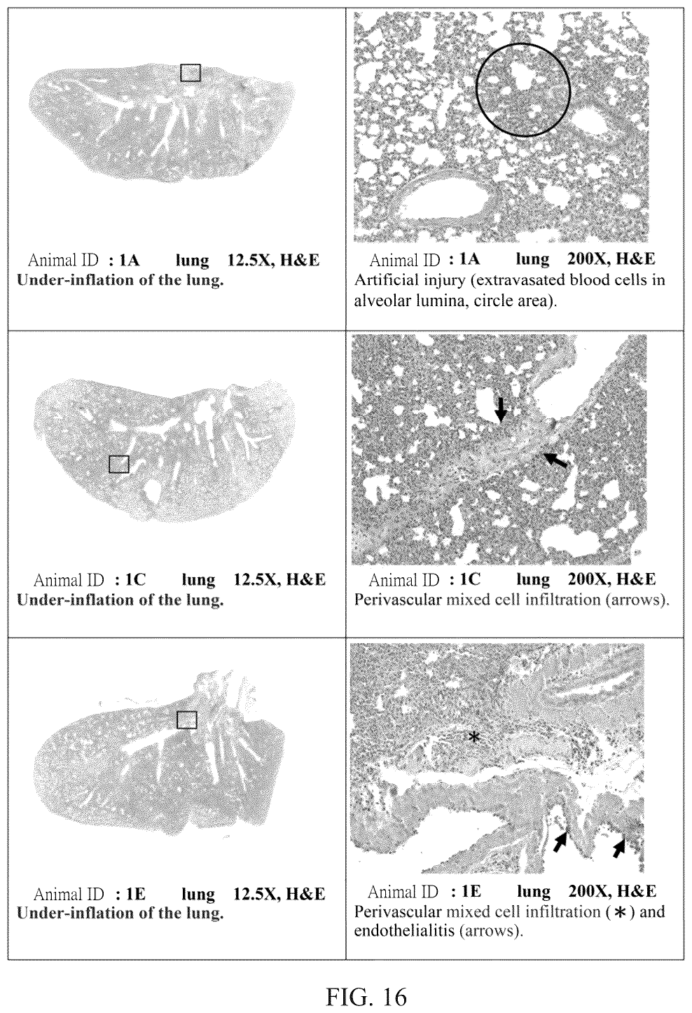

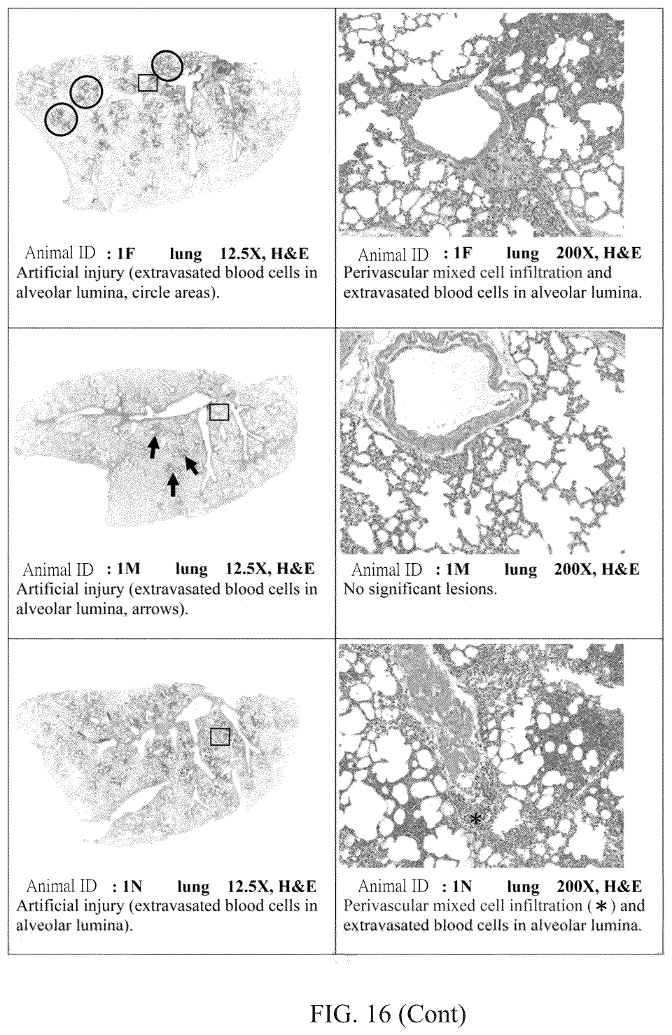

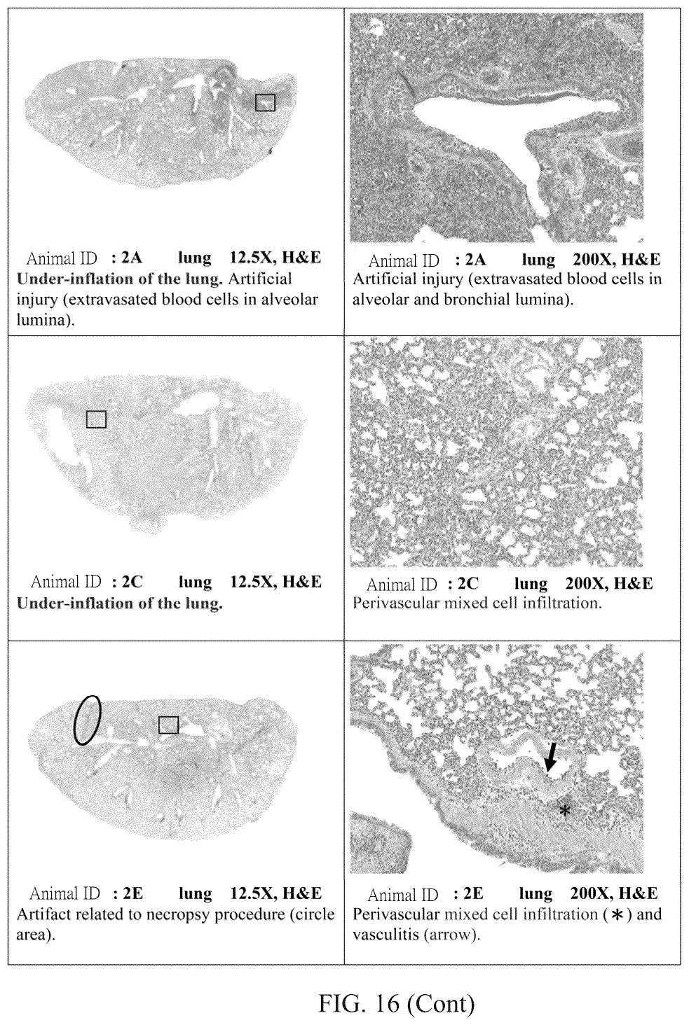

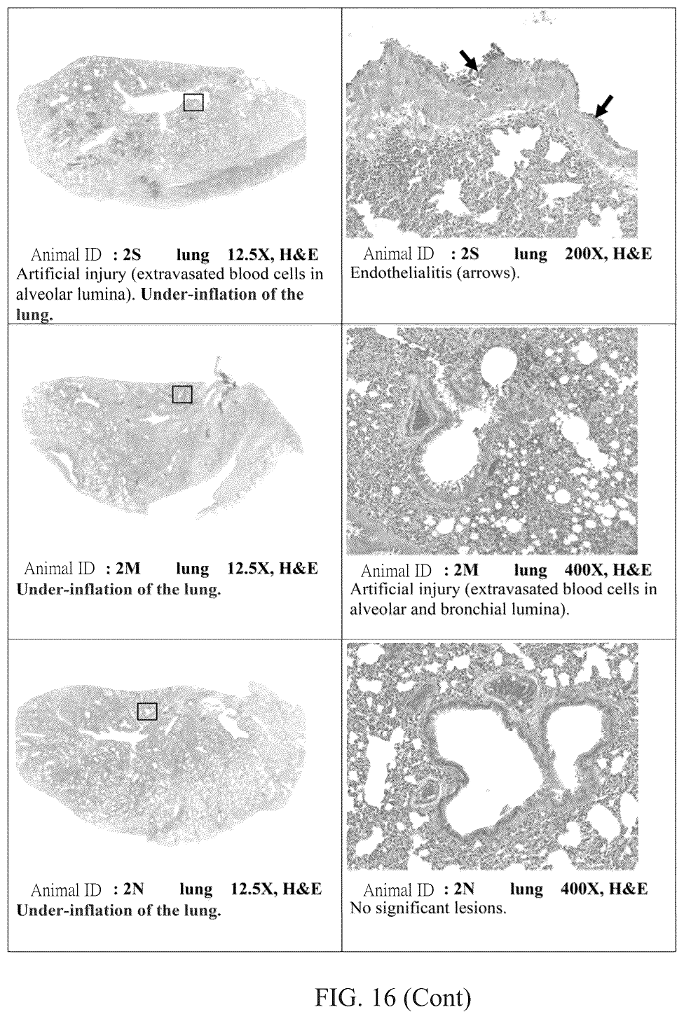

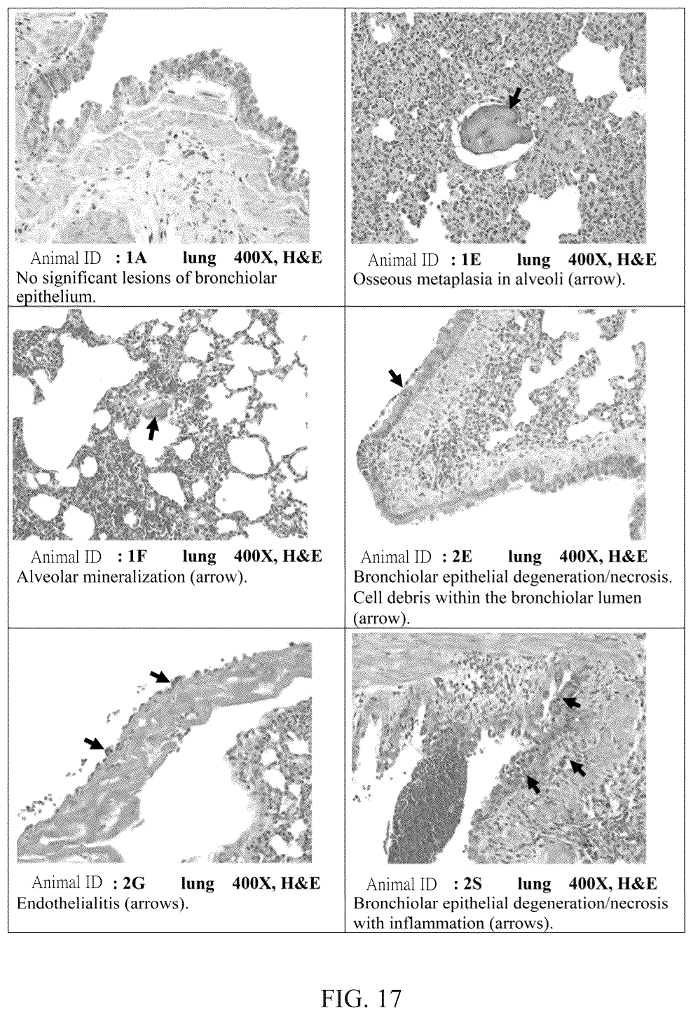

FIGS. 15A-15D show the Histopathology Finding Table in one animal experiment of SARS-CoV-2 omicron variant. FIG. 16 and FIG. 17 show the photographs of pathology examination in one animal experiment of SARS-CoV-2 omicron variant. The left column of FIG. 16 shows the lung tissue sections (12.5×) with H&E staining, and the right column shows the partial enlargement of the lung section, i.e. the higher magnification (200×) of the boxed area indicated in the left column. The animal sample ID and the observed symptoms were noted under each photograph. The animal ID and its detail information were shown in Table 2.

TABLE 2

| | | | | Number |
|---|---|---|---|---|
| | | | Day of post | of |
| Groups | Treatment | Animal ID. | inoculation | animals |
| 1 | SARS-CoV-2 | 1A, 1B, 1C, 1D | 2 | 4 |
| | omicron variant | 1E, 1F, 1G, 1H | 5 | 4 |
| | (placebo) | 1M, 1N, 1O, 1P | 10 | 4 |
| 2 | SARS-CoV-2 | 2A, 2B, 2C, 2D, 2R | 2 | 5 |
| | omicron variant | 2E, 2F, 2G, 2H, 2S | 5 | 5 |
| | and Test article | 2M, 2N, 2O, 2P | 10 | 4 |

Test article: Veldona (8 IU)

In one of the animal experiments of SARS-CoV-2 omicron variant, the histopathological examination results included the microscopic alterations presented in Histopathology Finding Table of FIGS. 15A-15D and the pathological photos presented in FIGS. 16-17. Histologically, Syrian hamsters infected with SARS-CoV-2 (Omicron variant) presented lesions including:

Mixed-Cellular Inflammation, Peribronchial Infiltration, and Perivascular Infiltration:

The lesions were characterized by a mixture of heterophils with lymphocytic and histiocytic cell types within alveoli/interstitial and peribrochial and perivascular in the lung. The lesion severity was minimal.

Bronchial Epithelial Cell Degeneration/Necrosis with or without Inflammatory Infiltration in Bronchiole:

The lesions were observed in lungs of SARS-CoV-2 inoculated animals and characterized by cellular swelling, cytoplasmic vacuolation, perinuclear clear spaces, pyknosis of nuclei of affected epithelium mixing up with heterophils infiltrate into the bronchial lumen. The lesion severity was minimal.

Vasculitis and Endothelialitis:

The lesions were observed in lungs of SARS-CoV-2 inoculated animals. Endothelialitis is characterized by blood vessel bulging into the lumen due to an infiltration by macrophages and lymphocytes. The lesion severity was minimal.

In addition, osseous metaplasia in alveoli and alveolar mineralization were observed in some submitted lungs. The lesion may be attributable to spontaneous or incidental lesion. Artificial injury related extravasated blood cells in alveolar lumina was considered as related to tissue sampling injury.

The SARS-CoV-2 inoculated related lesions including (1) Mixed-cellular inflammation, peribronchial infiltration, and perivascular infiltration; (2) Bronchial epithelial cell degeneration/necrosis with or without inflammatory infiltration in bronchiole; (3) Vasculitis and endothelialitis. Under-inflation of the lung and erythrocytes filled of the alveoli (artificial finding) were observed in most submitted lungs, histopathological examination was limited by these artifacts.

In this study, the effectiveness of the composition of the present application over a sixteen-day course (five-day pre-treatment and ten-day treatment after infection) of treatment of Omicron-variant-infected hamsters was evaluated. Compared with hamsters receiving solution without the test article (the “Control Group”), the hamsters receiving solution with the test article (the “Veldona Group”) demonstrated resistance to body weight loss immediately after infection, then showed a better recovery trend in the following three days. The body weights of the hamsters in the Veldona Group remained more stable than those of the hamsters in the Control Group during the treatment period.

For pathological indicators, on the tenth day, the hamsters in the Veldona Group showed none mixed-cellular inflammation, peribronchial infiltration, and perivascular infiltration, compared to 50% in the Control Group Hamsters in the Veldona Group in general showed promising results in treating indicators of new variant virus infection.

The virus infection is mainly in the upper respiratory tract, but in severe cases it will extend to the lower respiratory tract, that is, the lungs, causing serious irreversible complications. In this COVID-19 antiviral efficacy study, referring the results shown in FIGS. 1-3, 7, 8, 11A-11B, 12A-12D, 13 and 14, the delta variant-infected hamsters receiving the composition of the present application once-daily and orally were found to maintain significant body weight and recover similar to controls. After a 7-day course of treatment, Veldona group has effectively reduction of the virus. Combined with physiological properties and pathological findings, the results show that the composition of the present application having low-dose interferon in oral/sublingual formulation has a protective effect on the lung organs caused by SARS-CoV-2 by regulating the immune response. It also allows infected animals to return to normal physiological state more quickly. The composition of the present application is able to induce significant systemic immunomodulatory, has good anti-virus effect of Covid-19, and can be an effective treatment.

Referring the results shown in FIGS. 4-6, 9, 10, 15A-15D, 16 and 17, the omicron variant-infected hamsters receiving the composition of the present application once-daily and orally were found to maintain significant body weight and recover similar to controls. After a 16-day course of treatment, the Veldona group can effectively reduce the virus in this prophylactic study design. Combined with physiological properties and pathological findings, the results show that the composition of the present application has a protective effect on the lung organs caused by SARS-CoV-2 by regulating the immune response. It also allows infected animals to return to normal physiological state more quickly. The composition of the present application is able to induce significant systemic immunomodulatory, has good anti-virus effect of Covid-19, and can be an effective treatment.

In the present application, the composition is able to activate the self-immune system to achieve the effect of fighting against various viruses including SARS-COV-2. The composition can induce cytotoxicity and activate NK cells and antibody-dependent cytotoxicity. The composition comprising the recombinant human interferon against the coronavirus has obvious antiviral effects, and at the same time has great therapeutic potential in the prevention of the coronavirus infection.

While the present invention is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims and its equivalent systems and methods.

What is claimed is:

1. A method for treating and/or preventing an infection of Coronavirus comprising: providing a therapeutically effective amount of a composition comprising interferon to a subject via sublingual administration and/or buccal administration; wherein the Coronavirus comprises SARS-CoV-2.

2. The method of claim 1, wherein the composition comprises interferon alpha (INF-α).

3. The method of claim 2, wherein the INF-α is a recombinant INF-α.

4. The method of claim 2, wherein the composition comprises a dosage of equal to or less than 1,000 IU or 1,000 microgram of INF-α.

5. The method of claim 2, wherein the composition comprises a dosage of equal to or more than 1 IU of INF-α.

6. The method of claim 2, wherein the composition comprises a dosage of 1 IU -1,000 IU of INF-α.

7. The method of claim 1, wherein the composition further comprises a buffer, a carrier and/or an excipient.

8. The method of claim 2, wherein the composition further comprises interferon beta and/or interferon gamma.

9. The method of claim 2, wherein the composition further comprises an antiviral agent and/or anti-inflammatory agent.

10. The method of claim 1, which further comprises administering the subject with an antiviral agent and/or anti-inflammatory agent.

11. The method of claim 1, wherein the composition is in a dosage form of lozenge, tablet, film or spray.

12. A composition for treatment and/or prevention of an infection of Coronavirus comprises a therapeutically effective amount of interferon alpha (INF-α), wherein the composition is in a dosage form of sublingual administration and/or buccal administration.

13. The composition of claim 12, wherein the INF-α is a recombinant INF-α.

14. The composition of claim 12, wherein the INF-α is 1 IU-1,000 IU.

15. The composition of claim 12, which further comprises a buffer, a carrier and/or an excipient.

16. The composition of claim 12, which further comprises interferon beta and/or interferon gamma.

17. The composition of claim 12, which further comprises an antiviral agent and/or anti-inflammatory agent.

18. The composition of claim 12, which is in a dosage form of lozenge, tablet, film or spray.
